(19) United States
(12) Patent Application Publication
(10) Pub. No.: US 2008/0026083 A1
(43) Pub. Date: Jan. 31, 2008

(54) ANTIMICROBIAL AND PESTICIDAL COMPOSITIONS AND METHODS COMPRISING REDUCED MONOTERPENE OIL EXTRACTED FROM MYRTACEAE

(76) Inventor: Maxwell J. Reynolds, Queensland (AU)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/779,146

(22) Filed: Jul. 17, 2007

Related U.S. Application Data
(60) Provisional application No. 60/831,413, filed on Jul. 17, 2006.

(51) Int. Cl. A61K 36/00; A61P 43/00

(52) U.S. Cl. 424/725

(57) ABSTRACT

The invention comprises compositions and methods for the treatment or prophylaxis of a microbial, fungal, or mold infection in an animal, comprising the administration of a composition derived from the essential oil of a Myrtaceae plant, wherein at least about 85% of the monoterpene content of the oil has been removed, in an amount effective to prevent, reduce or eliminate the microbial, fungal, or mold infection.

### RELATED APPLICATIONS

This application claims priority from U.S. Provisional Patent Application No. 60/831,413, filed Jul. 17, 2006, which is hereby incorporated in full.

### FIELD OF THE INVENTION

The present invention relates to methods and compositions for preventing or controlling microbial infections or parasitic infestations in animals including mammals, insects, fish, crustaceans and birds.

### BACKGROUND

The present invention will be described with particular reference to the control and/or treatment of microbial, fungal and mold infections and parasitic infestations (collectively “microbial infections,” unless otherwise indicated by context) of farmed animals (land and marine), birds and beneficial insects such as bees. However, it will be appreciated that the methods and compositions described herein may also find application in the treatment and control of conditions in wild animals, fish, insects and birds and no limitation is intended thereby.

Microbial infections including bacterial and viral infections and parasitic infestations by agents such as acarids, protozoans and helminths in farmed animals and birds, beneficial insects, fish and crustaceans such as shrimp can have a significant economic impact on the relevant industries. Some conditions are untreatable and the only method of control is by culling. Other conditions may be treated with antimicrobial or pesticidal compositions. However, in many cases, the chemicals used in such treatments leave an unacceptable level of residue in a food product obtained either directly or indirectly from the animal, insect, bird or fish. This either prohibits the use of some active agents or restricts their use to particular window periods in the lifecycle. Further, many strains of infectious agents are becoming resistant to current antibiotics.

Exemplary conditions caused by infectious agents in animals include pneumonia, otitis media, sinusitus, bronchitis, tonsillitis, and mastoiditis related to infection by Streptococcus pneumoniae, Haemophilus influenzae, Moraxella catarrhalis, Staphylococcus aureus, Enterococcus faecalis, E. faecium, E. casselflavus, S. epidermidis, S. haemolyticus, or Peptostreptococcus spp.; pharyngitis, rheumatic fever, and glomerulonephritis related to infection by Streptococcus pyogenes, Groups C and G streptococci, Corynebacterium diphtheriae, or Actinobacillus haemo lyticum; respiratory tract infections related to infection by Mycoplasma pneumoniae, Legionella pneumophila, Streptococcus pneumoniae, Haemophilus influenzae, or Chiamydia pneumoniae; blood and tissue infections, including endocarditis and osteomyelitis, caused by S. aureus, S. haemolyticus, E. faecalis, E. faecium, E. durans, including strains resistant to known antibacterials such as, but not limited to, beta-lactams, vancomycin, aminoglycosides, quinolones, chloramphenicol, tetracylines and macrolides; uncomplicated skin and soft tissue infections and abscesses, and puerperal fever related to infection by Staphylococcus aureus, coagulase-negative staphylococci (i.e., S. epidermidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus aureus, coagulase-negative staphylococcal species, or Enterococcus spp.; urethritis and cervicitis; sexually transmitted diseases related to infection by Chlamydia trachomatis, Haemophilus ducreyi, Treponema pallidum, Ureaplasma urealyticum, or Neiserria gonorrheae; toxin diseases related to infection by S. aureus (food poisoning and toxic shock syndrome), or Groups A, B, and C streptococci; ulcers related to infection by Helicobacter pylori, systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellular; infections caused by Mycobacterium tuberculosis, M. leprae, M. paratuberculosis, M. kansasii or M. chelonei; gastroenteritis related to infection by Campylobacter jejuni; intestinal protozoa related to infection by Cryptosporidium spp.; odontogenic infection related to infection by viridans streptococci; persistent cough related to infection by Bordetella pertussis; gas gangrene related to infection by Clostridium perfringens or Bacteroides spp.; and atherosclerosis or cardiovascular disease related to infection by Helicobacter pylon or Chiamydia pneumoniae.

A particularly problematic condition which can occur in ruminants is paratuberculosis or Johne's disease. This is a contagious bacterial disease of the intestinal tract caused by the bacteria Mycobacterium paratuberculosis. The disease is a chronic condition contracted by calves but symptoms do not occur for several years. The disease causes a progressive thickening of the intestine and colon which slowly results in an irreversible loss of weight. There is no existing cure for this condition and infected animals must be slaughtered.

Parasitic diseases may be caused by either endoparasites or ectoparasites. Endoparasites are those parasites which live inside the body of the host, either within an organ (such as the stomach, lungs, heart, intestines, etc.) or simply under the skin. Ectoparasites are those parasites which live on the outer surface of the host but still draw nutrients from the host.

The endoparasitic diseases generally referred to as helminthiasis are due to infection of the host with parasitic worms known as helminths. Helminthiasis is a prevalent and serious worldwide economic problem due to infection of domesticated animals such as swine, sheep, horses, cattle, goats, dogs, cats, and poultry. Many of these infections are caused by the group of worms described as nematodes which cause diseases in various species of animals throughout the world. These diseases are frequently serious and can result in the death of the infected animal. The most common genera of nematodes infecting the animals referred to above are Haemonchus, Trichostrongylus, Ostertagia, Nematodirus, Cooperia, Ascaris, Bunostomum, Oesophagostomum, Chabertia, Trichuris, Strongylus, Trichonema, Dictyocaulus, Ca pillaria, Heterakis, Toxocara, Ascaridia, Oxyuris, Ancylostoma, Uncinaria, Toxascaris, and Parascaris. Many parasites are species specific (infect only one host) and most also have a preferred site of infection within the animal. Thus Haemonchus and Ostertagia primarily infect the stomach while Nematodirus and Cooperia mostly attack the intestines. Other parasites prefer to reside in the heart, eyes, lungs, blood vessels, and the like while still others are subcutaneous parasites. Helminthiasis can lead to weakness, weight loss, anaemia, intestinal damage, malnutrition, and damage to other organs. If left untreated these diseases can result in the death of the animal.

Infections by ectoparasitic arthropods such as ticks, mites, lice, stable flies, hornflies, blowflies, fleas, and the like are also a serious problem. Infection by these parasites results in loss of blood, skin lesions, and can interfere with normal eating habits thus causing weight loss. These infections can also result in transmission of serious diseases such as encephalitis, anaplasmosis, swine pox, and the like which can be fatal.

Animals may be infected by several species of parasite at the same time since infection by one parasite may weaken the animal and make it more susceptible to infection by a second species of parasite. Thus, a compound with a broad spectrum of activity is particularly advantageous in the treatment of these diseases.

Beneficial insects such as bees are also susceptible to infections and infestations which can have a significant commercial effect on the beekeeping industry. Bees are subject to infestations by mites such as varroa and acarine mites. Varroa mites, Varroa destructor are parasites that feed on the blood of the bees. Acarine mites, Acarapis woodi, infect the airways of the bee. Bees are also subject to bacterial infections such as American foulbrood and European foulbrood. These conditions are caused by bacterial infections of Bacillus larvae and Melisococus pluton respectively. American foulbrood is infectious and deadly to bee larvae.

Referring to treatment of mite infestation in bees, it has been known to use a variety of chemicals to fumigate the bee colonies, or to place certain other compounds therein to try to eliminate the mites from the hive where the colony resides, Among such materials are menthol, formic acid, bromopropylate, Coumaphos, pyrethrum extracts (both naturally occurring and synthetic types) and the like.

While generally useful, all of these compositions and techniques are not effective for a variety of reasons. One of the problems is that certain of these compounds, such as menthol and formic acid, when placed in the hive adversely affect the behavior of the bees. Because of their strong odor, the bees have an aversion to them and make every effort to remove them from the hive, With other compounds the bees must be removed from the hive, the hive treated, and the bee colony then returned after a period of several weeks. This is a costly and time-consuming process. Also, formic acid is corrosive and difficult and dangerous to handle. Moreover, certain of these compounds are only effective in warm weather conditions. This is particularly true with menthol, which requires at least two weeks of warm weather to cause it to vaporize in order to be effective. In many areas of t he world this is a condition that does not exist throughout the year and, thus, is not effective.

Moreover, with certain miticides it is difficult to cause the bees to ingest the same and this is particularly a problem in trying to treat tracheal mites residing in the trachea of the bees. If they cannot ingest the miticide to place it into their hemolymph, then the miticide will not be effective.

In addition to possible aversion to the treatment agent, there is also the problem of the need of high levels of usage which can have adverse effects on the beneficial insect. Here again, there is the need to ensure insect intake of the treatment agent, while at the same time minimizing the amount of agent used to minimize, and preferably eliminate, possible adverse effects.

In the case of honeybees, there is the further need to ensure that the treatment will not result in unacceptable levels of toxic chemicals in the hive products such as honey, beeswax, pollen, propolis, venom, and the like,

Another industry where conditions associated with infections and infestations can have significant economic impact is the aquaculture industry. The use of conventional antimicrobial and anti-parasitic compounds with fish and shrimp is that the build up of an unacceptable level of residue in the animal. Some conditions such as bacterial kidney disease and liver disease in salmon are effectively untreatable.

The poultry industry is yet another industry in which disease can have significant economic impact. The economic impact of avian flu has been particularly significant. The only treatment available for avian flu is culling.

It is therefore one object of the present invention to provide compositions and methods for treating or preventing infections and infestations in an animal, fish, bird or insect.

### SUMMARY

According to a first broad form of some embodiments of the invention, without limitation, there is provided a method for the treatment or prophylaxis of a microbial infection in an animal, the method comprising administering to said animal or locus thereof, an antimicrobially effective amount of a composition derived from essential oils of Myrtaceae plants, wherein at least about 85% of the monoterpene content of the oil has been removed.

According to a further broad form of some embodiments of the invention, without limitation, there is provided a method for the treatment or prophylaxis of a pest infestation in an animal, the method comprising administering to said animal or the locus thereof a pesticidally effective amount of a composition derived from essential oils of a Myrtaceae plant, wherein at least about 85% of the monoterpene content of the oil has been removed.

Other aspects of the invention will be apparent to those skilled in the art after reviewing the drawings and the detailed description below.

### DETAILED DESCRIPTION

In the present specification and claims, the term animal is to be construed widely as including any suitable animal within the kingdom animalia, and includes vertebrate animal such as mammals, birds and fish and also insects and crustaceans. The methods and compositions of the present invention are particularly applicable for the treatment and control of infections or infestations of domestic or agricultural or aquaculture animals.

The methods and compositions of the present invention will be described with particular reference to a composition derived from Melaleuca alternifolia. However, it will be appreciated that compositions of the invention may also be sourced from other Myraceae species and no limitation is intended thereby Other suitable sources of compositions of the present invention include, but are not limited to, M. Bracteata, M. Cricifolia, M. quinquinervia, and others appearing on Table 1, which is hereby incorporated in full by reference.

Essential oils are complex mixtures of volatile oils produced by plants and are responsible for the odor of many plants. The essential oil, once produced, is either released to the environment or stored in oil cells for later use. Essential oils stored in the wood of plants serves to deter micro-organisms and insects from attack.

Due to its being based on essential oils, its biofilm dissolving capacity has been established against Staphylococcus epidermidis, and its cidal effect against MRSA in vitro). We believe the broadband activity is due to all of these factors acting in unison to effectively dissolve biofilm, penetrate bacterial cell walls and then lyses, the bacteria (Mycobacterium tuberculosis).

Essential oils having antiseptic properties are well known. The essential oil obtained from the steam distillation of the stems and leaves of Myrtaceae family, of which one is Melaleuca afternifolia, is known as tea tree or Melaleuca oil. It is used widely as a topical antiseptic and in the control of ectoparasites such as fleas and head lice.

Essential oils contain large amounts of terpenes. Terpenes are classified according to the number of units of the basic structure methylbuta-1,3-diene or isoprene which make up the terpene. Monoterpenes contain two isoprene units and have the chemical formula C₁₀H₁₆. Terpenes may be acyclic such as myrcene and ocimene or cyclic such as limonene. Typically, commercially available Melaleuca oil comprises up to about 50% monoterpenes. Monoterpenes found in melaleuca oil include alpha-pinene, gamma terpinene, alpha terpinene and limonene.

Essential oils typically also contain sesquiterpenes. Sesquiterpenes contain three isoprene units and have the general formula C₁₅H₂₄ and are generally found in much lower quantities than the monoterpenes. For example, Melaleuca oil typically contains about 4 to 8% sesquiterpenes.

Another class of compounds commonly found in essential oils are known as oxygenates. These compounds have an oxygen containing functional group. Examples are aldehydes, phenol alcohols, carboxylic acids, ketones and esters. Terpin-4-ol, having the formula, C₁₀H₁₈O is a major constituent of Melaleuca oil and can constitute up to 40% of the oil. Terpin-4-ol is considered to be the major active constituent of Melaleuca oil. However, other oxygenated products and the monoterpenes are also believed to have some antimicrobial activity.

The composition of commercially available Melaleuca oils is partially regulated by International and Australian Standards. These standards set a minimum terpin-4-ol content of 30% and a maximum 1,8-cineol content of 15%.

Terpenes contain double bonds, which are susceptible to oxidation. It is believed that the capacity to generate activated oxygen intermediates may be responsible for their antimicrobial activity. On the other hand, this susceptibility to oxidation results in instability. Terpenes, particularly monoterpenes, are primarily saturated hydrocarbons, which are vulnerable to oxidation by oxygen in the environment surrounding the monoterpenes. The attack occurs in the region of the C—C double bonds of the terpene molecule. Such instability typically leads to discoloration, odor and premature loss of the proactive sites and also accounts for some of the observed heat sensitivity and chemic al reactivity of the essential oils. A further disadvantage is that some of these oxidation products may be irritating or even allergenic.

The present inventor has surprisingly and unexpectedly discovered that a composition derived from Melaleuca essential oil, whereby a major portion of the monoterpene content has been removed, not only exhibits improved stability but also retains and in some cases increases its antiseptic, antimicrobial and pesticidal properties. The present inventor has further observed that a preferred composition of the present invention exhibits improved antimicrobial and pesticidal properties when compared with conventional Melaleuca oil. Further still, the present inventor has discovered that whilst conventional Melaleuca oil is suitable only for topical administration in view of it's toxicity when ingested, that a preferred composition of the invention may be considered safe for oral administration in animals, birds, fish and insects and further does not leave an undesirable chemical residue in a food obtained from such organisms. Toxic effects, which may be experienced if after ingestion of commercial Melaleuca oil, include seizures, coma and respiratory depression.

The essential oil from which the composition is derived may be extracted from any one or more Myrdaceae species. Preferably the essential oil is extracted from Melaleuca alternifola. The essential oil is typically derived by known procedures such as steam distillation.

As discussed above, essential oils derived from Melaleuca species comprise a monoterpene fraction, an oxygenate fraction and a sesquiterpene fraction, although it will be appreciated that different species may contain different relative amounts of each fraction. Of these fractions, the monoterpenes are generally the most volatile and have the lowest molecular weight. Thus, they may be removed by techniques known to those of skill in the art including vacuum low temperature techniques such as inert gas flushed distillation; molecular weight separation techniques including chromatographic techniques and selective solvent extraction techniques. Prefer ably, the monoterpenes are removed under reduced pressure and at a temperature which does not exceed 50° C., preferably 40° C. Typically between about 80 and about 99% of the monoterpenes are removed, typically between about 90 and about 99%.

A preferred composition for use with the methods of the invention is derived from the essential oil of Melaleuca altemifolia and typically comprises from between about 40 to about 60%, preferably between about 50 to about 55% terpen-4-ol and between about 8 to about 30%, preferably between about 8 to about 25% sesquiterpenes. The sesquiterpene fraction may include aromadendrene, voridiflorene, delta cadinene, globulol and/or viridiflorol.

The antimicrobial composition may find application as an antibacterial, antiprotozoan, antifungal and/or antiviral agent. Typically, the composition is effective against a broad range of micro-organisms including E. coli, S. aureus, P. aeruginosa, C albicans, S. epidermidis, Penicilium ssp, Cladosporium, A. Niger, A. fumigatus, P. expansum, S. chartarum, Alteraria, Aspergillus, Fusarium, B. subtills, B. cereus, C. perfringens, K. pneumoniae, L. lactis, M. smegmatis, S. marcescens, S. pyogenes, A. viridans, E. aerogenes, S. liquefaciens, P. vulgaris, S. enteridis, P. mirabills, S. abaetetuba, L. monocytogenes, N. Gonorrhoeae, Legionella, M. Gordanoae and M. catarrhalis and viruses including coronavirus, rotavirus, adenovirus, herpes simplex, papillovirus, rhinovirus, hepatitis B and A, enterovirus and parainfluenza virus.

Bacterial infections and protozoal infections, and disorders related to such infections, which may be treated or prevented in animals, birds, fish and insects, include the following: bovine respiratory disease related to infection by P. haemolytica, P. multocida, Mycoplasma bovis, or Bordetella spp.; cow enteric disease related to infection by E. coli or protozoa (i.e., coccidia, cryptosporidia, etc.); dairy cow mastitis related to infection by S. aureus, Strep. uberis, Streptococcus agalactiae, Streptococcus dysgalactiae, Corynebacterdum, or Enterococcus spp.; swine respiratory disease related to infection by A. pleuro., P. multocida, or Mycoplasma spp.; swine enteric disease related to infection by E. coli, Lawsonia infracellularis, Salmonella, or Serpulina hyodysinteriae; cow footrot related to infection by Fusobacterium spp.; cow metritis related to infection by E coli; cow hairy warts related to infection by Fusobacterium necrophorum or Bacteroides nodosus; cow pink-eye related to infection by Moraxella bovis; cow premature abortion related to infection by protozoa (i.e. neosporium); urinary tract infection in dogs and cats related to infection by E. coli; skin and soft tissue infections in dogs and cats related to infection by S. epidermidis, S. intermedius, coagulase neg. Staphylococcus or P. multocida; and dental or mouth infections in dogs and cats related to infection by Alcaligenes spp., Bacteroides spp., Clostridium spp., Enterobacter spp., Eubacterium, Peptostreptococcus, Porphyromonas, or Prevotella, American and European foul brood in bees caused by Bacillus larvae and Melissococcus pluton, chalkbrood in bees, a fungal disease associated with Ascophera apis and Nosema in bees caused by a protozoan Nosema apisl; bacterial kidney disease (BKD) in salmon, Enteric Redmouth Disease (ERM) of salmon as caused by the bacterial pathogen Yersinia ruckeri, and, Vibriosis a bacterial disease of salt-water and migratory fish caused by the bacterium Vibrio anguillarum or Vibrio salmonicida, known as a cold water vibriosis which affects Atlantic salmon.

Viral infections and disorders associated with such infections which may be treated or prevented in animals and birds include the following: bovine herpesvirus 1-5 (BHV), ovine herpesvirus 1 and 2, Canine herpesvirus 1, equine herpesvirus 1-8 (EHV), feline herpesvirus 1 (FHV), and pseudorabies virus (PRV), porcine herpesvirus (PRV) or Aujeszky's disease (pseudorabies virus or PRV), the porcine reproductive respiratory syndrome virus (or PRRSV), the swine influenza virus (SIV), the conventional hog cholera virus (or HCV), parvoviruses in porcines, bovine respiratory syncitial virus (BRSV), bovine diarrhoea virus (BVDV), porcine reproductive respiratory syndrome virus (PRRSV), swine influenza virus (SIV), rabies virus, hog cholera virus, porcine parvoviruses (HCV), herpesvirus of turkeys (HVT), Infectious Bovine Rhinotracheitis (IBR), Avian Infectious Bronchitis Virus (IBV), Avian Influenza Virus, Fowlpox-Virus, Avian Infectious Laryngotracheitis Virus (AILV), Mycoplasma Marek's Disease Virus (MDV), Newcastle Disease Virus (NDV), Avian Paramyxovirus Type 1, Avipoxvirus Isolates such as Juncopox, Pigeon Pox, and Field—(Field) and vaccine strains of bird pox viruses, Avian Encephalomyelitis Virus, Avian Sarcoma Virus, Rotavirus, Avian Reovirus and H7 Influenza Virus.

The present invention also relates to a method of controlling a pest infestation in an animal, bird, fish or insect. Pests which may be controlled include ecto and endoparasites. Examples of pests which may be controlled by the method of the present invention include gastrointestinal worms such as liver fluke, hookworm, heartworm whipworm and tapeworm; ticks, mites, lice and flies.

The composition may be in any suitable form and for internal or external use. Preparations for internal use include powders, tablets, dispersible granules capsules, solutions, suspensions, and emulsions suitable for oral ingestion or injection. In a preferred embodiment, the composition is provided in the form of a food supplement which may be added to an animal's food or water supply.

The composition may also find use as a topical anti-microbial and/or anti-parasitic agent. Examples of such applications include antiseptic scrubs or washes and flea and lice shampoos, spray, plunge dips and pur-on formulations.

The topical compositions may also be administered in the form of wound dressings, transdermal patches and the like. Typically, although not exclusively, wound dressings are impregnated with a composition of the invention at a concentration of active agents of between about 10000 to about 5000 ppm.

In a further application, the composition may be applied in the form of an aerosol. Such formulations may be used for the treatment of lung infections.

In preferred embodiments, without limitation to only those disclosed herein and without disclaimer of other embodiments, effective dosing may be determined in accordance with the disease and the level of infection encountered. As some examples only, and without limitation, if the disease is detected early in the infection, one may use doses of between 5-10 mg per kilo body weight. If the disease is detected when the infection has taken a strong hold, one may use between 50-100 mg per kilo body weight. As a prophylactic dose to prevent disease, i.e. influenza, one may use dose levels of 1-5 mg per kilo body weight.

In the methods of the invention, the composition may also be applied to the locus of an animal, fish, bird or fish. Where the composition is used to treat infections and infestation in fish the composition may also be in the form of an additive which may be added to the water in a fish or holding tank.

The composition may also be used in the form of a fumigant for the purposes of controlling infectious agents in an animal enclosure such as a dairy, stable, pen or the like. A particular application is the fumigation or fogging of bee hives to control or treat mite infestations.

Without limiting embodiments of the invention to only those expressly disclosed herein and without disclaiming other embodiments, Table 2 describes further treatment details and methods that comprise some embodiments of the invention. For purposes herein, including without limitation, for Table 2 and the examples below, “base composition” means the composition of Example 3 below or reasonable equivalents thereof.

### EXAMPLES

The following examples are provided without limiting the invention to only those embodiments described herein and without disclaiming other embodiments.

### Example 1

Without limitation to any particular process, a basic process for manufacture of Melaleuca alternifolia essential oil is as follows:

The plants are harvested from a field by cutting the scrubs off just above ground level. They are mulched and placed into a steel trailer-like transport vessel. The trailer is brought into a distillation shed. Steam is pushed through the mulched wood and leaf mix, and water and oil is collected via a stainless steel condenser. The oil is centrifuged out of the water oil mix and collected. The oil is then cleaned up and concentrated for the final product.

### Example 2

In some embodiments, without limitation to only those embodiments expressly disclosed herein, essential oil from Melaleuca alternifolia oil has the following characteristics:
Product Name: Oil of Melaleuca alternifolia
Synonyms: Tea Tree Oil; Melaleuca Oil; Oil of Melaleuca, Terpinen-4ol type
Definition. Essential oil steam distilled from the leaves and terminal branchlets of Melaieuca alternifolia
Chemical Abstract No.: 68647-73-4
UN No.: 2319
Tariff CN Code: 3301.29.61
   Physical Properties:
Appearance: Clear, mobile liquid with no visible trace of water
Color: Colorless to pale yellow
Odor: Characteristic warm, spicy odor
Specific Gravity@20 degreesC/20 degrees C: 0.885-0.906
Refractive Index@20 degrees C: +5o to +15o
Solubility@20 degrees C: 1 vol soluble in 2 vols 85% v/v ethanol

Chemical Properties:

| | | |
|---|---|---|
| Analysis: | 1,8 Cineole 3% +/.1% | Terpinen-4-ol 39% +/.2% |

Typical analysis by gas chromatography

| Constituent | Proportion | ISO Standard |
|---|---|---|
| alpha-pinene | 2.4% | 1-6% |
| sabinene | 0.6% | trace-3.5% |
| alpha-terpinene | 10.2% | 5-13% |
| p-cymene | 2.2% | 0.5-12% |
| Limonene | 1.1% | 0.5-4% |
| 1,8-cineole | 3.6% | 0-15% |
| gamma-terpinene | 20.5% | 10-28% |
| alpha-terpinolene | 3.6% | 1.5-5.0% |
| terpinene-4-ol | 40.2% | 30%+ |
| alpha-terpineol | 3.1% | 1.5-8% |
| aromadendrene | 1.1% | trace-7% |
| delta-cadinene | 1.0% | trace-8% |
| globulol | 0.3% | trace-3% |
| viridiflorol | 0.3% | trace-1.5% |

### Example 3

Without limitation, some embodiments of the invention comprise a composition prepared by removing essentially all the monoterpene fraction from Melaleuca altemifola essential oil. The composition of said oil after removal of the monoterpene fraction is about as follows:

Test Results

| | Weight | |
|---|---|---|
| Compound | PerCent* | CAS No. |
| Para cymene | 0.34 | 000535-77-3 |
| Alph Terpinene | 1.15 | 000099-85-4 |
| Linalool | 0.37 | 029050-33-7 |
| 2-cycohexen-1-ol,1-methyl- | 0.27 | 029803-82-5 |
| 4-1(1-methyethyl)-,cis | | |
| 2/cyclohexen-1-ol,1-methyl-4- | 0.68 | 029803-81-4 |
| 91-methylethyl | | |
| 2-cylohexan-1-ol,methyl- | 0.53 | 029803-82-5 |
| 4-(1-methylethyl)-cis | | |
| Terpinene-4-ol | 48.90 | 000562-74-3 |
| Alpha terpineol | 9.04 | 000098-55-5 |
| 2-cyclohexen-1-ol,3-methyl- | 0.49 | 016721-38-3 |
| 6-(1-m-ethylethyl)-cis | | |
| Cyclohexanol,2,3,5- | 0.41 | 000116-02-9 |
| trimethyl | | |
| 2,4-heptadiene,2,6-dimethyl- | 0.42 | 004634-87-1 |
| Sorbic acid | 0.39 | 000110-44-1 |
| Viriflorol | 0.37 | 1000156-10-8 |
| Cubebene | 0.64 | 017699-14-8 |
| Isoeugenyl methyl ether | 0.26 | 000093-16-3 |
| 2-cyclopenten-1-one,3,4,5- | 0.19 | 055683-21-1 |
| Trimethyl | | |
| Beta caryophyelline trans | 1.11 | 000489-40-7 |
| Caryophyllene | 1.08 | 000087-44-5 |
| 1h-cyclopropa(a)naphthalene | 0.28 | 020071-49-2 |
| alloaromadendrene | 4.39 | 025246-27-9 |
| 111-cycloprop(e)azulene | 1.01 | 126362-40-7 |
| 1a,2,3,4,ra,5,6,7b-octahydro-1,4,7 | | |
| tetrame | | |
| Alpha Carophyllene | 0.43 | 006753-98-6 |
| Aromadendrene | 1.71 | 000489-39-4 |
| Isolendene | 1.31 | 1000156-10-8 |
| Naphthalene,1,2,3,4,4a,5,6,8a- | 0.97 | 005951-61-1 |
| Hexahyd | | |
| Gama Gurguene | 4.04 | 021747-46-6 |
| Azulene,1,2,3,4,5,6,7,8-octahydro- | 2.79 | 000088-84-6 |
| 1,4-dimethyl-7-(1-methylethylidene) | | |
| Alpha thugene | 4.72 | 000483-76-1 |
| Naphthalene,1,2,3,4-tetrahydro-1 | 2.13 | 000483-77-2 |
| 6-dimethyl-4-(1-methylethyl) | | |
| Alpha cubenbene | 0.77 | 017699-14-8 |
| Lipiglobulol | 0.66 | 100150-05-1 |
| Tricyclo(6.3.0.1)undec-7-ene | 0.40 | 1000152-25-6 |
| 6,10,11,11-tetramethyl | | |
| Spathulenol | 0.96 | 077171-55-2 |
| Globulol | 1.47 | 1000150-05-0 |
| III-cycloprop9c)azulen-4-ol | 1.40 | 000552-02-3 |
| IH Indene, | 0.78 | 056324-68-6 |
| 1-ethylideneoctahydro-7 | | |
| Naphthalene,1,2,3,4,4a,7-hexahydro- | 1.49 | 016728-99-7 |
| 1,6-dimethyl-4-(1-methylethyl | | |
| Viridflorene | 0.92 | 058569-25-8 |
| Copaene | 0.73 | 003856-25-5 |
| *General analysis as determined by Gas Liquid Chromatography Mass Spectrophotometer analysis. Those of ordinary skill in the art will appreciate that such determinations may vary between or among test machines and conditions based on factors which include, but are not limited to, column material, temperature of injection port, oven temperature, and/or detection system. Thus, the formulation above is exemplary without limiting | | |
| # the embodiments to only those listed and without disclaiming other embodiments not specifically listed herein. | | |

Physical Properties:
Color—Pale Yellow
Aroma—Medicinal
Clarity—Clear
Viscosity—1.080
Specific Gravity—0.943
Boiling point—approx. 148 degrees C
Density by weight—0.9259

### Example 4

Use of the base composition was tested in conjunction with the control of avian H5N1 virus.

One day old chicks were supplied a water soluble solution of the base composition at a 10% concentration level. This was a stock solution which was broken down with water to 200 ppm or 0.02%. The diluted solution was fed to the chickens from day one until 48 hours prior to slaughter and dressing. Findings indicated that the body weight will increase from traditional feeding systems; as a consequence, time of housing to kill may decrease and that will depend upon target finish weight, i.e., in Australia they aim for 3.2 kilos average live chicken.

For already growing chickens, a fogging system was developed to protect the birds from the virus before the change over to new 1 day old birds.

The fogger delivered about 10 to 30 micron droplets of 1% base composition solution, with about 2 liters per 25,000 birds per spray. All parts of the animal storage shed were sprayed as well as the birds so that they inhale the test solution into their systems, so offering not only a virus free environment but also the base composition transferred into the blood system via the lungs of the growing birds. The sheds and litter were fogged prior to the birds being placed in the sheds or in the case of established sheds, the birds are fogged daily for the first week and then weekly.

A protocol for chicken treatment to prevent avian H5N1 virus infection was established. Other trials have shown that concentrations of birds deliberately infected with a range of levels of virus produce results from death to sick birds, to no reaction to the virus introduction.

The virus H5N1 was grown in fertile eggs titer established and then by serial dilution made into the following concentrations of virus:
1. 10×6
2. 10×5
3. 10×4
4. 10×3
5. 10×2

In the cases of birds infected at 1 and 2 all were dead in 36 hours.

In the case of level 3, one bird died in 36 hours; two birds were found dead on day 4; one bird found dead on day 6, and one bird survived for 11 days but was terminated although it may have recovered.

In both cases 4 and 5, the birds showed no signs of clinical distress.

In case 3 antibodies were detected while in cases 4 and 5 no antibodies were found.

The study indicated that the birds infected to levels of 10×4 plus had virus take hold and kill the birds but at varying rates.

The fact that no antibodies were located in cases 4 and 5, i.e., 1000×100 cells per ml ingestion levels suggested that the virus could not establish a growth rate sufficiently to cause infection or death in the birds.

From live bird trials, we have established that feeding chicks from day one at a level of 200 ppm of the base composition in their water bred health chickens. The studies also showed that both male and female birds reached a body weight at 56 days of about 4.3 kilograms, high for normal birds and occurring in both sexes. Although these birds were not exposed to the avian virus it does show a high rate of food conversion to body weight donating healthy birds.

This further indicates that if the viral infection of the birds is low to begin with, around 10×4 that daily uptake of the base solution in the drinking water may stop or depress the growth rate of the virus, allowing the birds to survive, and produce antibodies to further attack and kill the virus if they do become infected.

Combining a fogging system at 1% base composition in the sheds to kill or reduce virus and other bacteria (e.g., S. aureus, E. Coli, etc.) should contain all infections both viral and bacterial and without the use of antibodies. This would have the added advantage of speeding up the growth time of the birds, and improve the turn round time in the sheds, with cost-effective reduction in feed, use and housing time, and the like.

### Example 5

The antimicrobial activity of a composition comprising some embodiments of the invention was examined. The base composition was tested using a 96-well microtiter tray and the concentrations of the base composition were in the range of about 2-0.0025%.

Food pathogens were prepared in double-strength Mueller Hinton broth, resulting in the organisms at final concentrations between 1.5×10⁵-2.5×10⁶ cfu/ml. Tests were incubated at 35° C. for 24 hours and subcultured by removing 10 microliters from tray wells and spot inoculating onto blood agar. All subcultures were incubated and the colonies counted. The minimum inhibitory concentration (“MIC”) was defined as the lowest concentration of product resulting in the maintenance or reduction of the inoculum. The minimum cidal concentration (“MCC”) was defined as the lowest concentration of product resulting in the death of 99.9% of the inoculum. The tests were carried out twice. If discrepant results were obtained, a third test was conducted.

The MlCs of strains of Campylobacter jejuni were determined by agar dilution using Mueller Hinton Agar containing 5% lysed horse blood. The inocula were prepared in 0.85% saline and a volume of 10 ul/spot was applied onto the MHA plates. The final concentration was approximately 5×105×1 Os cfu/spot. All results were read after 48 h of incubation in a 37° C. in a microaerophilic atmosphere (5% 02, 10% CO2, 85% N2). The MIC was defined as the lowest giving complete inhibition of visible growth. See Table 3 below for results.

### Example 6

A composition comprising some embodiments of the invention was tested, starting at 10% base concentration, against 20 clinical isolates.

Testing on Vancomycin resistant Enterococcus faecium and faecalis (“VRE”) bacteria as well as MRSA, major problem in some medical facilities.

We found that golden staph bacteria were inhibited at 0.12% base composition; VRE bacteria were inhibited at 1% concentration of base composition; and ESBL bacteria were inhibited at 0.5% concentration of base composition.

One embodiment was tested against 20 clinical isolates of multi-resistant methicillin resistant Staphylococcus aureus using agar dilution techniques. The tested concentrations initially were 0.03% and 0.06% base composition. All 24 clinical strains including ATCC controls grew at both concentrations, Agar dilution testing was repeated using additional concentrations ranging from 0.015% to 2% base composition. Secondary testing was performed.

Complete inhibition of all clinical and control samples was detected at a concentration of 0.25% v/v base composition. 58% (14/24) of the clinical strains were inhibited at a concentration of 0.12% v/v base composition. All clinical isolates and ATCC control strains of Staphylococcus aureus were inhibited than a concentration of 0.25% v/v.

A 10% solution of the base composition was tested against 20 clinical isolates of the vancomycin resistant Enterococcus species using an agar dilution technique.

13 vancomycin resistant strains of Enterococcus faecium and seven vancomycin resistant strains of Enterococcus faecalis were tested. Organisms were selected from a time period spanning five years to minimize the potential for testing of clonal isolates. 14 strains exhibiting the vanB genotype (high level to vancomycin resistance plus teicoplanin susceptibility) were tested compared to six strains with the vanA genotype (high level vancomycin and teicoplanin resistance). This distribution reflects the predominance of the vanB Enterococcus faecium in nosocomial outbreaks in Australia.

Our studies found complete inhibition of all clinical and control strains of Enterococcus species at a concentration of 1% v/v. All clinical excellence and ATCC control strains of vancomycin resistant Enterococcus were inhibited at a concentration of 1% v/v irrespective of resistance genotyping species.

Mycobacterium avium subspecies paratuberculosis (M. paratuberculosis) is a very slow growing mycobactin dependent mycobacterial species known to be the causative agent of Johne's disease (paratuberculosis) in all species of domestic ruminants. The bacteriostatic activity (growth inhibition) of the base composition was tested on following mycobacteria:

M. paratuberculosis (strains CLIJ62, CLIJ748, ATTCJ9698)—slow growing mycobacterial species with lengthy growth period in vitro on solid based laboratory media and a more rapid growth cycle in a selective broth environment;

M. avium (strain D4)—a moderate growing mycobacterial species; and
M. smegmatis (strain mc²155)—a fast growing non-pathogenic mycobacterium

The BACTEC™ 460 radiometric system is a well established semi-automated broth-based culture system procedure that provides rapid detection of M. paratuberculosis. The BACTEC procedure for antimicrobial susceptibility testing of mycobacteria is based on the same basic principal used in the conventional method: for bacterial propagation a liquid medium is used instead of counting colonies after a long incubation period, the growth is monitored radiometrically, and results are reportable within a shorter time frame. The amount of ¹⁴CO₂ released from ¹⁴C-labelled substrate reflects the rate and amount of growth occurring in the vial and is expressed in terms of the “Growth Index” (“GI”). Bacteria were considered:

Susceptible: when GI was completely abolished during the course of experiment;

Inhibited: when GI was partially (temporarily) inhibited during the course of experiment;

Resistant: when was not affected during the course of experiment.

The bacteriostatic activity (“growth inhibitions) of the base composition for mycobacteria in the active phase of growth was evaluated in vitro using the BACTEC 460 radiometric culture system using three concentrations of base composition solution supplied. The MICs determined for M. paraetuberculosis, M. avium and M. smegmatis were 0.1%; 0 125%^{r} and 0.15% base composition. Growth inhibition was dependent on the inoculum size, source, growth rate and incubation period. We concluded that:

the base composition is able to affect the bacterial metabolism of all three mycobacteria species tested at concentrations low as 1% of the 10% base composition solution;

M. paratuberculosis appeared most susceptible to the base composition;

unrestricted growth of each bacterial inoculum was observed in all culture vials in the absence of antimicrobial agent or diluent (vitamin E solution);

acid fastness detected by Ziehl Neelsen staining of the mycobacterial suspensions confirmed the purity of all cultures and the presence or absence of mycobacteria; and

growth inhibition depends on the activity of the inoculum, the rate of growth and the incubation period.

### Example 7

The acute oral toxicity of the base composition was investigated in 4 Sprague Dawley Specific Pathogen Free (“SPF”) rats at doses of 500 and 1000 ppm base composition at 10 mL/kg. The experimental procedure was based on OECD guidelines for the testing of chemicals No. 401.

The test solution was administered orally once to 2 pairs of rats at the above doses. A third group was administered the vehicle only, solubilized vitamin E solution. The equivalent volumetric dose was 10 ml/kg for all groups.

Body weights were determined immediately before test item administration and at sacrifice on day 8. All animals were observed at frequent intervals on the day of test item administration and then daily for signs of toxicity over the 7 day experimental period, at the end of the experimental period, all animals were sacrificed and subjected to a gross necroscopy examination.

Results showed that no mortalities were observed during the study; no clinical abnormalities were observed for the duration of the study in any of the treated or control animals; and there were no gross abnormalities noted in the major organs of any animal at necroscopy. Further analysis was carried out by gas chromatography testing of the kidneys and livers. No traces of the components of the base composition were found, indicating that all compounds are successfully excreted from the body.

Based on the results obtained from this study, the base composition, up to the highest doses tested 1000 ppm at 10 ml/kg, did not produce toxicity in the Sprague Dawley rat. These results may be compared to tea tree oil which has been reported to have an LD₅₀ of 1.9-2.6 ml/kg.

### Example 8

Acute oral toxicity testing in rats revealed no signs of toxicity at tested levels.

In one study, the acute oral toxicity of 10% solution of base composition was investigated in groups of 2 Sprague Dawley Specific Pathogen Free (SPF) rats at a single dose level. The test solution was administered orally by intragastric gavage to one (1) group of 2 rats (1/sex) at the maximum administrable volume of 10 mL/kg. A control group of 2 rats (1/sex) was dosed with the vehicle alone (water). Body weights were determined immediately before test item administration and daily thereafter. All animals were observed at frequent intervals on the day of test item administration, and then daily for signs of toxicity over the 7-day experimental period. At the end of the experimental period, on Day 8, all surviving animals were sacrificed and subjected to a gross necropsy examination. There were no deaths or abnormal clinical signs in any animal during the experimental period. Overall body weight gains occurred in all animals. There were no gross abnormalities found in the major organs of any animal treated with the vehicle or test item at autopsy. Under the conditions of this study, the tested 10% solution produced no signs of toxicity when administered at a volume of 1 mL/kg.

### Example 9

Dermal toxicity studies in guinea pigs revealed no signs of toxicity at tested levels.

Thirty Duncan-Hartley Guinea Pigs (25 females and 5 males) were ranked by weight and sex and then randomly allocated into five groups of females (n=5 per group) and one group of males (n=5). The treatment groups met the regulatory requirements of having the No Observable Effects Limit (“NOEL”) group, the Lowest Observable Effects Limit (“LOEL”) group, and the Mean Dose Lethality (MDL) or Maximum Toxicity Dose (“MTD”) group.

On Day 0 of the study, Groups 1 (NOEL) and 3 (MTD/MDL) were treated with a 5%, 15%, and 25% formulation of base composition. Group 4 received treatment with 100% base composition and served as an extreme positive control. Group 5 received the vehicle, consisting of a specifically formulated Vitamin E base and served as a primary negative control. Treatment was administered via a treated gauze pad containing 2 ml of the test substance applied directly to a 2 cm square shaved are on the dorsum of each guinea pig. This provided a direct application of 1 ml/cm². The five male guinea pigs were additional to Group 3 and met the requirement of having on test a group of alternate sex subjects receiving the MTD/MDL dose.

Health scores were documented for each individual guinea pig twice daily. Observations were more frequent on the day of treatment. Particular attention was paid to immunogenic, vasodilatory or necrotizing changes of the treatment area. Post mortem examinations were performed on all animals at the end of the study, as none had died on test and any gross abnormalities were documented on a Necropsy Record. Histological examination was conducted on skin tissues taken from the treatment area from each group. Had any gross abnormalities been observed on necropsy, these tissues would have been submitted for histopathological examination. There were no abnormalities identified and therefore this procedure was not performed.

Abnormal findings occurred in those animals on test designated as Group 4 or also identified as the positive control group. It was anticipated that necrotizing changes would occur within this group. None of the animals in the test dose groups had been affected and therefore it was determined that there was no toxic effect when administered cutaneously to guinea pigs at the 5%, 15% and 25% levels as an acute study for toxicity. Gross pathology, and health scores were basically normal for the animals on test. Only those animals in the positive control group demonstrated any abnormal findings as was anticipated. There was no evidence of an induced response between those animals receiving the test substances and those receiving the negative control substance resulting in an identifiable or measurable difference of any significance. Weight gain analysis demonstrated a strong equivalence amongst all groups over the duration of the test including the positive control group suggesting that any adverse changes were local and not systemic in nature. The overall finding of the study was that the base composition, when administered as a single bolus to guinea pigs on an acute toxicity test by dermal application, did not demonstrate any increasing toxic effect corresponding to dosage.

### Example 10

The effect of the base composition on microbial infections was tested in vitro. In microbiological testing, plates were prepared using Tryptic Soy Agar (bacteria) and Malt Extract (yeast and mould).

All microorganisms were tested against 108 (MacFarland Standard) concentration of micro organism, etc. The MacFarland turbidity standard system was used to estimate the level of bacteria in a solution by the opacity of the liquid against a known MacFarland tube purchased from a biological supplier

The results are shown in the following Table:

| | No pf tests | Average zone of |
|---|---|---|
| | carried | inhibition/mm |
| E. Coli | 105 | 24 |
| S. aureus | 68 | 30 |
| P. aeruginosa | 48 | 22 |
| C albicans | 60 | 35 |
| S epidermidis | 30 | 38 |
| Penicillium ssp | 68 | Ng |
| Cladosporium | 89 | Ng |
| A. Niger | 65 | Ng |
| A. fumigatus | 5 | Ng |
| P. Expansum | 5 | Ng |
| S. chartarum | 4 | Ng |
| Alternaria | 10 | Ng |
| Aspergillus | 5 | Ng |
| Fusarium | 4 | Ng |
| B. subtilise | 15 | 35 |
| B. cereus | 20 | 35 |
| C. perfringens | 5 | Ng |
| K. pneumoniae | 2 | Ng |
| L. lactis | 1 | Ng |
| M. Smegmatis | 5 | Ng |
| S. Macescens | 3 | Ng |
| S. pyogenes | 3 | Ng |
| A. viridans | 10 | Ng |
| E. aerogenes | 2 | Ng |
| S. liquefaciens | 2 | Ng |
| P. vulgaris | 15 | Ng |
| S. enteridis | 4 | Ng |
| P. mirabilis | 5 | Ng |
| S. abaetetuba | 15 | Ng |
| L. monocytogenes | 20 | Ng |
| N. Gonorrhoeae | 5 | Ng |
| Legionella | 10 | 35 |
| M. Gordanoae | 14 | 35 |
| M. catarrhalis | 3 | Ng |

ng denotes no growth

### Example 11

The efficacy of the base composition in inactivating H5N1 virus in an egg test environment was tested. Work was done to confirm the ability of the base composition to act as a virucidal agent against a Vietnamese H5N1 highly pathogenic avian influenza virus strain.

A 10% solution of the base composition (Batch 020405) was made in dimethyl sulphoxide (DMSO) by adding 1.0 ml of base composition to 9.0 ml of DMSO and mixing thoroughly. A solution of 1% base composition with no emulsifiers (Batch 102034) was supplied (NeuMedix Ltd, Underwood, Queensland, Australia) and used as supplied for virus treatment.

The virus was A/chicken/Vietnam/8/2004 H5N1 grown in the allantoic sac of embryonated, SPF chicken eggs. Infectious allantoic fluid was harvested, pooled and stored at −80° C. for this trial. The microstores pool reference number of this material is 0404-30-1550.

The 10% base composition was diluted 1:5, 1.3.3 and 1:2.5 in phosphate buffered saline (pH 7.3) to give final concentrations of 2%, 3% and 4% respectively. Based on ASTM E1052-96, 0.1 ml of virus was mixed with 0.4 ml of base composition at concentrations of 2%, 3% and 4%. Mock-treated virus, consisting of 0.1 ml of virus mixed with 0.4 ml of a 1:5 dilution of DMSO without added base composition in PBS and untreated virus, consisting of 0.1 ml of virus mixed with 0.4 ml of PBS, were also prepared. All mixtures were incubated at room temperature for the respective times. 2%, 3% and 4% base composition solutions were diluted 1:10 in PBS and 0.1 ml inoculated into 5 eggs each.

Virus Titrations

Trial 2—in SPF Eggs

Residual virus was assayed by making 10-fold dilutions in PBS of each virus mixture from 10⁻¹ to 10⁻⁸0.02 ml of virus mixture was added to 1.8 ml of PBS and mixed thoroughly to give a 10⁻¹ dilution. 0.2 ml of this was added to 1.8 ml of PBS and mixed thoroughly to give a 10⁻² dilution and so on to a final dilution of 10⁻⁸0.01 ml of each dilution was inoculated into the allantoic sac of embryonated, SPF chicken eggs, incubated at 37° C. for 3 days or until embryo death. Eggs were examined twice daily for viability. At death or after 3 days incubation all eggs were chilled overnight at 4° C. and then tested for the presence of hemagglutination and an indicator of virus infection. The residual infectivity tire was calculated by the method of Reed, and Meunch.

Results

The base composition treatments and egg inoculations for this trial showed no egg mortalities due to the effects of the virus inoculum.

Summary of Titration Results of Trial 3:

Log 10 residual virus titer after base composition treatment in (EID⁵⁰/0.1 ml)

| Base composition | Treatment Time | | |
|---|---|---|---|
| concentration | 60 Minutes | 120 Minutes | 240 Minutes |
| 2% | 4.1 | 4.5 | 3.1 |
| 3% | 2.9 | 3.5 | 2.9 |
| 4% | 3.1 | 2.7 | 0 |
| Untreated virus | 7.1 | | |
| Mock-treated virus | 7.5 | | |

From the results presented above, we concluded that the base composition can inactivate between 0.3 and 7.1 log 10 of H5N1 avian influenza virus depending on the base composition concentration and contact time between the virus and base composition. A 7.1 log 10 inactivation means that all virus was killed. Eggs inoculated with base composition solutions alone showed no egg deaths.

### Example 12

Analysis by electron microscopy was conducted to evaluate changes in the ultrastructure of the virus after administration of the composition.

A solution of base composition in phosphate buffered saline (pH 7.3) to give a final concentration of 4%. Mock-treated virus, consisting of 0.1 ml of virus mixed with 0.4 ml of a 1:5 dilution of DMSO without added base composition in PBS and untreated virus, consisting of 0.1 ml of virus mixed with 0.4 ml of PBS, were also prepared. All mixtures were incubated (three hours, 180 mins) at room temperature for the respective times. Concentrations and times were selected in accordance with experience from previous experiments. The parameters were selected as they resulted in significant reduction in virus titer:

TABLE 4

| | Treatment Time | | |
|---|---|---|---|
| Test concentration | 60 Minutes | 120 Minutes | 240 Minutes |
| 2% | 4.1 | 4.5 | 3.1 |
| 3% | 2.9 | 3.5 | 2.9 |
| 4% | 3.1 | 2.7 | 0 |
| Untreated virus | 7.1 | | |
| Mock-treated virus | 7.5 | | |

Log 10 residual virus titer after base composition treatment in (EID₅₀/0.1 ml)

Solutions were prepared for negative contrast electron microscopy as per the protocol described in the QA manual. All samples were imaged with a Philips CM 120 at 100 kK and recorded with a MegaView III digital camera.

Upon treatment of H5N1 virus with 4% base composition for three hours, the ultrastructure of the virus changes. Positive control samples displayed ultrastructure consistent with that described for viruses belonging to the family Orthomyxoviridae (Virus Taxonomy. Eight Report of the International Committee on Taxonomy of Viruses. Edited by C. M. Fauqet, M. A. Mayo, J. Maniloff, U. Desselberger, L.a. Bail. Elsevier, Academic Press, 2005, pgs. 681-693). The ultrastructure of H5N1 treated as described differed in that the stain penetrated the membrane envelope and the majority of surface projections were absent. The ability of the stain to penetrate a greater proportion (subjective observation) of viruses following treatment may be indicative of disruption to the envelope.

To gain an appreciation of the significance of the above changes it should be noted that the surface membrane incorporates various viral proteins and support the surface projections (which are required for infection of host cells) whilst encompassing the viral nucleic acid (nucleocapsid) which is required for replication. One interpretation of the ultrastructural changes could be the inference that they are consistent with the data in Table 4 which show a substantial rise in viral inactivation when H5N1 is exposed to a 4% concentration of base concentration for a contact time of greater than 120 mins.

### Example 13

Bees

Varroa Mites

Trial 1

Extensive field trials have been conducted on 2500 bee hives. The level of mite infection was estimated by catching individual bees and counting the number of mites thereon. Without any treatment at all, the average number of mites per 50 bees was 10.

About 70 to about 90 grams of the base composition at a concentration of 1000 ppm in food grade mineral oil as a carrier was fogged into each hive. Fogging was conducted initially for three consecutive days and then every 10 days all year round.

Treatment with the inventive composition reduced the mite infestation level to about 1 mite per 50 bees.

Comparative trials were conducted with food grade mineral oil on its own and also mixtures of eucalyptus, lavender, and commercially available tea tree oil with food grade mineral oil under the conditions recited above. The level of mite infestation after these comparative treatments was about 5 mites per 50 bees.

It may be seen that these field trial results indicate that the composition of the present invention is superior to current methods of varroa mite control in bee hives.

Trial 2

In the light of the positive field trial results, a further controlled trial is to be conducted as outlined below:

A honey bee colony will be selected with at least 50 varroa mites per 300 bees. Approximately 300 bees will be placed in each of 30 metal cages (105×100×40 mm with mesh on one side and glass on the other. Honey will be provided on the floor of the cage on an upturned lid. Additional food will be provided in the form of 2M sugar syrup.

Ten cages of bees will not be treated as negative controls. The base composition will be mixed with food grade mineral oil at 500 and 1000 ppm. This will then be applied to the bees through the mesh on the cage using a fogger. Each treatment and rate will be replicated ten times. The actual application amount will be calculated by weighing the cages before and after fogging.

After fogging, the number of mites to fall to the bottom of the cages will be counted after 4, 24 and 48 hours. After 48 hours, the bees w ill be removed, counted and washed in alcohol to remove any live mites.

European Foulbrood

European foul brood (EFB) is a disease of honey bee larvae caused by Melissococus pluton, a gram positive bacteria. EFB kills larvae at about 3 days of age but doe not affect adult bees. From the results given above under the heading Microbiological testing, it would be expected that a base composition would be effective in the control of EFB.

It is proposed to determine the minimum inhibitory concentration of the base composition of Example 3 to M. pluton. When this concentration has been determined, trials will be conducted with infected bee colonies to illustrate the effectiveness of the composition of Example 3 against EFB.

All attachments, tables, and cited references are hereby incorporated by reference in their entireties as though fully set forth herein.

It will be appreciated that various changes or modifications may be made to the invention as described and claimed herein without departing from the spirit and scope thereof. While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

TABLE 1
Plants of the Myrtaceae Family

Acmena smithii (Lilly-Pilly Tree)
Agonis flexuosa (Peppermint Tree, Australian Willow Myrtle)
Agonis juniperina (Juniper Myrtle)
Angophora costata (Sydney Red Gum, Smooth-Barked Apple)
Astartea fascicularis
Baeckea virgata
Callistemon citrinus (Lemon Bottlebrush, Crimson Bottlebrush)
Callistemon linearis (Narrow-leafed Bottlebrush)
Callistemon rigidus (Stiff Bottlebrush)
Callistemon salignus (White Bottlebrush)
Callistemon viminalis (Weeping Bottlebrush)
Callistemon viminalis ‘Little John’ (Callistemon ‘Little John’, Dwarf
Bottlebrush)
Calothamnus quadrifidus
Calothamnus villosus (Silky Net Bush, Woolly Net Bush)
Calytrix tetragona (Fringe Myrtle)
Chamelaucium uncinatum (Geraldton Waxflower)
Eucalyptus albens (White Box)
Eucalyptus caesia (Gungurru)
Eucalyptus camaldulensis ssp. camaldulensis (River Red Gum)
Eucalyptus camaldulensis ssp. obtusa (Murray River Red Gum,
Red Gum)
Eucalyptus campaspe (Silver Topped Gimlet)
Eucalyptus cinerea (Ash-Colored Eucalyptus)
Eucalyptus citriodora (Lemon Scented Gum)
Eucalyptus cladocalyx (Sugar Gum)
Eucalyptus conferruminata (Bushy Yate)
Eucalyptus cornuta (Yate)
Eucalyptus deglupta (Mindanao Gum)
Eucalyptus eremophila (Tall Sand Mallee, Horned Mallee)
Eucalyptus erythrocorys (Red Cap Gum, Illyarrie, Bookara Gum)
Eucalyptus erythronema (Red-flowered Mallee)
Eucalyptus ficifolia (Red Flowering Gum, Scarlet Gum)
Eucalyptus formanii (Forman's Mallee)
Eucalyptus globulus (Tasmanian Blue Gum)
Eucalyptus gracilis (Yorrell)
Eucalyptus grossa (Coarse-flowered Mallee)
Eucalyptus gunnii (Cider Gum)
Eucalyptus incrassata (Lerp Mallee)
Eucalyptus kruseana (Kruse's Mallee)
Eucalyptus largiflorens (Black Box)
Eucalyptus leucoxylon (White Iron Bark)
Eucalyptus macrocarpa (Mottlecah)
Eucalyptus maculata (Spotted Gum, Spotted Iron Gum)
Eucalyptus mannifera ssp. maculosa (Red-spotted Gum)
Eucalyptus megacornuta (Warted Yate)
Eucalyptus melliodora (Yellow Box)
Eucalyptus microtheca (Coolibah)
Eucalyptus nicholii (Willow Peppermint)
Eucalyptus niphophila (Snow Gum)
Eucalyptus orbifolia (Round-leafed Mallee)
Eucalyptus papuana (Ghost Gum)
Eucalyptus pauciflora (Cabbage Gum)
Eucalyptus perriniana (Spinning Gum, Round Leaved Snow Gum)
Eucalyptus platypus (Round-leafed Moort)
Eucalyptus polyanthemos (Silver Dollar Gum)
Eucalyptus populnea (Bimble Box, Poplar Box)
Eucalyptus preissiana (Bell-fruited Mallee)
Eucalyptus pulverulenta (Silver Mountain Gum)
Eucalyptus pyriformis (Pear-fruited Mallee)
Eucalyptus rhodantha (Rose Mallee)
Eucalyptus robusta (Swamp Mahogany)
Eucalyptus rudis (Desert Gum, Flooded Gum)
Eucalyptus saligna (Sydney Blue Gum)
Eucalyptus sargentii (Salt River Mallet)
Eucalyptus sideroxylon (Red Ironbark)
Eucalyptus spathulata (Narrow Leaf Gimlet, Swamp Mallee)
Eucalyptus stellulata (Black Sally)
Eucalyptus tetraptera (Square-fruited Mallee)
Eucalyptus torelliana (Cadaga)
Eucalyptus torquata (Coral Gum)
Eucalyptus viminalis (Manna Eucalyptus)
Eucalyptus woodwardii (Lemon Flowered Gum, Lemon Flowered Mallee)
Eugenia aggregata (Cherry of the Rio Grande)
Eugenia apiculata (Palo Colorado)
Eugenia brasiliensis (Grumichama)
Eugenia jambos (Rose Apple, Pomarrosa)
Eugenia luschnathiana (Pitomba)
Eugenia uniflora (Surinam Cherry, Pitanga)
Feijoa sellowiana (Feijoa, Pineapple Guava)
Kunzea baxteri (Scarlet Kunzea)
Leptospermum laevigatum (Coastal Tea Tree)
Leptospermum petersonii (Lemon Tea Tree)
Leptospermum rotundifolium
Leptospermum scoparium (New Zealand Tea Tree)
Lophostemon confertus (Brisbane Box, Vinegar Tree, Brush Box)
Melaleuca alternifolia (Tea Tree, Snow in Summer)
Melaleuca armillaris (Drooping Melaleuca, Bracelet Honey Myrtle)
Melaleuca decussata (Lilac Melaleuca)
Melaleuca diosmifolia (Diosma-Leaved Honey Myrtle, Green Honey
Myrtle)
Melaleuca elliptica
Melaleuca ericifolia (Heath Melaleuca, Swamp Paperbark)
Melaleuca hypericifolia (Dotted Melaleuca)
Melaleuca incana (Gray Honey Myrtle)
Melaleuca linariifolia (Snow-in-Summer, Flax-leaved Paperbark)
Melaleuca megacephala
Melaleuca nesophila (Showy Honey Myrtle, Pink Melaleuca)
Melaleuca quinquenervia (Cajeput Tree)
Melaleuca styphelioides (Prickly Paperbark, Prickly-leaved Tea Tree)
Melaleuca wilsonii
Metrosideros excelsus (Pohutukawa, New Zealand Christmas Tree)
Metrosideros karmadecensis (Kermadec pohutukawa)
Mosiera ehrenbergii
Myrciaria cauliflora (Jaboticaba)
Myrtus communis (True Myrtle, Common Myrtle, Roman Myrtle)
Myrtus communis ‘Boetica’ (Twisted myrtle)
Pimenta dioica (Allspice, Pimento, Jamaica pepper)
Pimenta racemosa (West Indian Bay Tree, Bay Rum Tree)
Psidium cattleianum (Strawberry Guava)
Psidium cattleianum ssp. lucidum (Lemon Guava)
Psidium guajava (Tropical Guava)
Psidium guineense (Brazilian Guava, Wild Guava)
Syzygium aromaticum (Clove)
Syzygium francisii (Giant Water Gum)
Syzygium paniculatum (Brush Cherry)
Syzygium samarangense (Java Apple, Makopa)
Tristaniopsis laurina (Kanooka, Water Gum)
Ugni molinae (Chilean Guava)

Adapted from http://www.desert-tropicals.com/Plants/Myrtaceae/Myrtaceae.html.

TABLE 2

| | | |
|---|---|---|
| 1. Disease | Paratuberculosis | |
| Formulation | | |
| Active | Base composition | 2.0% |
| Base Carrier | Water | 97.8% |
| Emulsifier | Vitamin E | 0.2% |
| | Total | 100.0% |
| Delivery System | Slow dissolving capsule | |
| Delivery Load | 50 mg per kilo Body weight | |
| 2. Disease | American Foulbrood Bee | |
| Formulation | Base composition | 1.0% |
| | Food Grade Mineral Oil | 99% |
| | Total | 100% |
| Delivery System | Fogger Butane type | |
| Delivery Load | Fog the hive three times to start | |
| | and then very 10 days | |
| 3. Disease | European Foul Brood Bees | |
| | As Above | |
| 4. Disease | Respiratory infections including the following: | |
| | Mycoplasma pneumoniae | |
| | Legionella pneumoniae | |
| | Streptococcus pneumoniae | |
| | Haemophlus influenza | |
| | Chiamydia pneumoniae | |
| Formulation | Base composition | 0.1% |
| | Base carrier Canola oil | 99.9% |
| Delivery System | Liquid Canola Oil | |
| Delivery Load | 1 ml per kilo/body weight | |
| 5. Disease | Blood and tissue Infections including the following: | |
| | Endocarditis, S. auerus, S hamolyticus, E fascalis | |
| | Osteomyelitis, E durans, beta-lactams vancomycin. | |
| Formulation | Base composition | 0.10% |
| | Base carrier saline water | 99.89 |
| | Vitamin E | 0.01% |
| Delivery System | Intravenous Injection | |
| Delivery Load | 1 ml per kilo body weight | |
| 6. Disease | Soft Tissue cuts and wound and abscesses | |
| | Caused by the following bacterial infections: | |
| | S aureus, streptococcus agalactiae, viridans streptococci | |
| | Corynebactereium minutissimun, Clostridium spp, | |
| | Bartonella henselea | |
| Formulation | Base composition | 1.0% |
| | Base Carrier Soy Oil PH Gr | 99.0% |
| Delivery System | Spray-Gel-Cream | |
| Delivery Load | As required | |
| 7. Disease | Parasitic disease caused by endoparasities or ectoparasites such as | |
| | Helminthiasas, Trichostrongylus, Ostertagia, Nematrodris, Cooperia, | |
| | Ascaris, Bunostomum. | |
| Formulation | Base composition | 1.0% |
| | Base Carrier water | 98.9% |
| | Vitamin E | 0.1% |
| Delivery System | Capsule 1 gram | |
| Delivery Load | 1 Capsule per 10 kilo body weight | |
| 8. Disease | Infections by ectoparasitic arthropods such as | |
| | ticks, mites, lice, stable flies, hornflies, blowflies, fleas. | |
| Formulation | Base composition | 2.0% |
| | Base Carrier Soy Oil | 98.0% |
| Delivery System | Spray-Gels-Creams | |
| 9. Disease | Avian Influenza | |
| Formulation | Base composition | 0.02% |
| | Water | 99.96% |
| | Vitamin | 0.002% |
| Delivery System | Drinking water Prevention System | |
| Delivery Load | As required: | |
| | Chick will drink twice what they eat a day | |

TABLE 3

% v/v

| Species | MIC | MCC |
|---|---|---|
| Aeromonas hydrophila ATCC7966 | 0.01 | 0.01 |
| Bacillus cereus ATCC11778 | 0.03 | 0.06 |
| Clostridium perfringens ATCC12915 | 0.03 | 0.01 |
| Enterococcus faecalis NCTC8213 | 0.125 | 0.25 |
| Escherichia coli ATCC 25922 | 0.06 | 0.06 |
| Escherichia coli ATCC105365 | 0.03 | 0.03 |
| Enterbacter aerogenes ATCC13048 | 0.06 | 0.125 |
| Klebsiella pneumoniae ATCC13883 | 0.03 | 0.03 |
| Listeria monocytogenes ATCC35152 | 0.03 | 0.06 |
| Listeria monocytogenes 001/05 | 0.06 | 0.125 |
| Listeria monocytogenes 002/05 | 0.06 | 0.125 |
| Listeria monocytogenes 007/05 | 0.06 | 0.06 |
| Listeria monocytogenes 010/05 | 0.06 | 0.06 |
| Listeria monocytogenes 011/05 | 0.03 | 0.125 |
| Listeria monocytogenes 013/05 | 0.03 | 0.125 |
| Listeria monocytogenes 323/01 | 0.03 | 0.125 |
| Listeria monocytogenes 327/04 | 0.06 | 0.125 |
| Listeria monocytogenes 328/04 | 0.06 | 0.25 |
| Listeria monocytogenes NCTC 7923 | 0.06 | 0.125 |
| Proteus vulgoris NCTC4635 | 0.03 | 0.03 |
| Serratia odorifera ATCC33077 | 0.06 | 0.125 |
| Shigella flexneri ATCC12022 | 0.03 | 0.03 |
| Shigella sonnei ATCC25931 | 0.03 | 0.03 |
| Staphylococcus. aureus ATCC9144 | 0.03 | 0.06 |
| Staphylococcus aureus ATCC33592 | 0.03 | 0.06 |
| Streptococcus agalactiae ATCC13813 | 0.005 | 0.005 |
| Streptococcus bovis ATCC9809 | 0.06 | 0.125 |
| Vibrio cholera M3695 | 0.01 | 0.01 |
| Vibrio parahaemolyticus ATCC43996 | 0.01 | 0.01 |
| Vibrio vulnificus ATCC29306 | 0.01 | 0.01 |
| Yersinia enterocolitica ATCC27729 | 0.03 | 0.06 |
| Campylobacter jejuni NCTC11351 | 0.06 | |
| Campylobacter jejuni 361/04 | 0.06 | |
| Campylobacter jejuni 362/04 | 0.06 | |
| Campylobacter jejuni 384/04 | 0.06 | |
| Campylobacter jejuni 387/04 | 0.06 | |
| Campylobacter jejuni 38/05 | 0.06 | |
| Campylobacter jejuni 27/05 | 0.06 | |
| Campylobacter jejuni 405/04 | 0.06 | |
| Campylobacter jejuni 412/04 | 0.06 | |
| Campylobacter jejuni 413/04 | 0.06 | |
| Campylobacter jejuni 416/04 | 0.06 | |
| Salmonella Salford WACC1 | 0.125 | 0.125 |
| Salmonella Typhimurium 0532 | 0.06 | 0.06 |
| Salmonella Typhimurium 018331 | 0.06 | 0.06 |
| Salmonella Infantis 017431 | 0.06 | 0.125 |
| Salmonella Singapore 0402 | 0.06 | 0.06 |
| Salmonella Livingstone 017424 | 0.06 | 0.06 |
| Salmonella Livingstone 011626 | 0.06 | 0.125 |
| Salmonella Livingstone 011655 | 0.06 | 0.125 |
| Salmonella Liverpool 10674 | 0.06 | 0.06 |
| Salmonella Cerro 012823 | 0.06 | 0.125 |
| Salmonella species ATCC12915 | 0.06 | 0.06 |

What is claimed is:

1. A method for the treatment or prophylaxis of a microbial, fungal, or mold infection in an animal, comprising the step of administering to said animal or locus thereof in an amount effective to prevent, reduce, or eliminate the infection, a composition derived from the essential oil of a Myrtaceae plant wherein at least about 85% of the monoterpene content of the oil has been removed.

2. A method for the treatment or prophylaxis of a pest infestation in an animal, the method comprising administering to said animal or the locus thereof in an amount effective to prevent, reduce, or eliminate the infestation, a composition derived from the essential oil of a Myrtaceae plant wherein at least about 85% of the monoterpene content of the oil has been removed.

3. The method of claim 1 or claim 2, wherein the Myrtaceae plant is Melateuca alternifolia.

4. The method of claim 1 or claim 2, wherein the monoterpenes have been removed under reduced pressure at a temperature of less than 50° C.

5. The method of claim 1 or claim 2 in which the composition comprises between about 40 to about 60% terpen-4-ol and between about 8 to about 30% sesquiterpenes.

6. The method of claim 1, wherein the microbe is a bacterium and the animal is a ruminant.

7. A method for the treatment or prophylaxis of paratuberculosis in a ruminant, the method comprising administering to the ruminant, an effective amount of a composition derived from the essential oil of a Myrtaceae plant, wherein at least about 85% of the monoterpene content of the oil has been removed.

8. A method for the treatment of prophylaxis of a mite infestation in a honeybee colony, the method comprising administering to the locus of the colony a vaporised composition derived from the essential oil of a Myrtaceae plant, wherein at least about 85% of the monoterpene content of the oil has been removed.
